(19) United States
(12) Patent Application Publication
(10) Pub. No.: US 2026/0125517 A1
(43) Pub. Date: May 7, 2026

(54) IODINATED MULTI-FUNCTIONAL COMPOUNDS WITH ENHANCED HYDROLYTIC STABILITY IN RADIOPAQUE HYDROGELS

(71) Applicant: Boston Scientific Scimed, Inc., Maple Grove, MN (US)

(72) Inventors: Joseph Thomas Delaney, JR., Minneapolis, MN (US); Cristian Parisi, Amherst, MA (US); Yen-Hao Hsu, Shrewsbury, MA (US); Rick William Dorn, Plymouth, MN (US); Nicolas Ball-Jones, Minneapolis, MN (US)

(73) Assignee: Boston Scientific Scimed, Inc., Maple Grove, MN (US)

(21) Appl. No.: 19/380,155

(22) Filed: Nov. 5, 2025

Related U.S. Application Data
(60) Provisional application No. 63/717,396, filed on Nov. 7, 2024.

(51) Int. Cl. C08J 3/075; A61K 49/00; A61K 49/04

(52) U.S. Cl. CPC C08J 3/075; A61K 49/0073; A61K 49/0442; C08J 2300/102; C08J 2300/104; C08J 2300/106

(57) ABSTRACT

In some aspects, the present disclosure provides a hydrolytically stable iodinated multi-functional compound comprising: a multi-functional compound having functional groups that are reactive with a reactive group of a multi-arm polymer; an iodinated polyol having reactive moieties including two or more hydroxyl groups, and one or more iodinated aromatic groups; and a linker between a reactive moiety of the iodinated polyol and a functional group of the multi-functional compound, where the linker includes a carbamate linkage or a carbonate linkage.

### CROSS-REFERENCE TO RELATED APPLICATIONS

This application claims the benefit of U.S. Provisional Patent Application Ser. No. 63/717,396 filed on Nov. 7, 2024, the disclosure of which is incorporated herein by reference.

### FIELD

The present disclosure relates to iodinated multi-functional compounds for forming radiopaque hydrogels (e.g., radiopaque hydrogel networks), more specifically to iodinated multi-functional compounds with enhanced hydrolytic stability in the radiopaque hydrogels and methods of formation of the iodinated multi-functional compounds. The radiopaque hydrogels and systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as reactive end groups which further react with trilysine acetate (TLA) to form crosslinks. This product has become a very successful, clinically used biomaterial in prostate cancer therapy. A further improvement based on this structure is that a portion of the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups will rapidly react with the trilysine crosslinker in vivo to form a hydrogel. The hydrogels breakdown in-vivo over the course of ca. 6-9 months. The breakdown occurs primarily through the hydrolysis of the ester linkages on the glutarate groups.

Future versions of SpaceOAR® products family may include formulations containing iodine atoms onto the trilysine molecule structure. When enhanced hydrolytic stability of the covalent bond connecting the trilysine to the iodinated species may be desired, ester linkages may not be desired. However, many iodinated species, and especially commercialized and approved iodinated contrast agents, contain alcohols which can undergo straightforward condensation/esterification reactions with the carboxylic acid group of trilysine. Nonetheless, inclusion of these approved iodinated contrast agents is desired as they are readily available, manufactured on large scales, well-studied from a toxicology/biological standpoint, and commonly used in clinical practice.

### SUMMARY

The present disclosure provides an alternative approach to the SpaceOAR® products described above. For instance, in some embodiments herein a linker or moiety is employed in conjunction with contrast agents (e.g., iodinated polyols) that has increased hydrolytic stability, as compared to a hydrolytic stability of the most hydrolytically labile group (e.g., glutaric ester linkages) employed with respect to other components (e.g., a multi-arm polymer) in the SpaceOAR® products described above. Thus, the resultant rate of hydrolysis of a crosslinked gel network in a hydrogel herein is higher than the rate of hydrolysis of the contrast agent-to-crosslinker to maintain radiopacity of the cross-linked gel network (e.g., at least until hydrolysis of the most hydrolytically labile groups) and hence improves performance (e.g., imaging performance remains unchanged or suitable for a longer time) of the crosslinked gel network. That is, the linkers herein (e.g., heterobifunctional linkers) can include a reactive moiety or functional group (e.g., isocyanates, chloroformate esters, etc.) that can form hydrolytically stable covalent bonds with traditional components such as iodinated polyols (e.g., iodixanol, etc.) that remain hydrolytically stable longer than hydrolysable bonds (e.g., those formed via the glutaric ester linkages) in the hydrogel. Yet, the linkers herein (e.g., heterobifunctional linkers) also include a reactive moiety or functional group (e.g., an amine) that can couple with traditional components such as traditional cross-linkers (e.g., trilysine) which permit the formation of the crosslinked gel networks via the traditional cross-linkers.

In some aspects, a hydrolytically stable iodinated multi-functional compound comprises a multi-functional compound having functional groups that are reactive with a reactive group of a multi-arm polymer; an iodinated polyol having reactive moieties including two or more hydroxyl groups, and one or more iodinated aromatic groups; and a linker between a reactive moiety of the iodinated polyol and a functional group of the multi-functional compound, wherein the linker includes a carbamate linkage or a carbonate linkage.

In some aspects, a radiopaque hydrogel is formed by covalently crosslinking (a) a hydrolytically stable iodinated multi-functional compound comprising: a multi-functional compound having functional groups that are reactive with a reactive group of a multi-arm polymer; an iodinated polyol having reactive moieties including two or more hydroxyl groups, and one or more iodinated aromatic groups; and a linker between a reactive moiety of the iodinated polyol and a functional group of the multi-functional compound, wherein the linker includes a carbamate linkage or a carbonate linkage, with (b) a multi-arm polymer comprising a core, a plurality of polymer segments having a first end that is covalently attached to the core and a second end comprising a moiety that comprises a reactive group that is reactive with the functional groups of the hydrolytically stable iodinated multi-functional compound.

A kit for forming a radiopaque hydrogel comprising: a reactive multi-arm polymer (e.g., having a functionality of three or more) comprising a core, a plurality of polymer segments having a first end that is covalently attached to the core and a second end comprising a moiety that comprises a reactive group; a multi-functional compound (e.g., crosslinker with a functionality of two or more) that comprises functional groups that are reactive with the reactive group of the reactive multi-arm polymer; an iodinated polyol having two or more hydroxyl groups, and one or more iodinated aromatic groups; and a heteropolyfunctional linker comprising a first terminal functional group that is reactive with a hydroxyl group of the iodinated polyol and at least one second terminal functional group that is reactive with the functional groups of the multi-functional compound to form a linkage between the iodinated polyol and the multi-functional compound, wherein the heteropolyfunctional linker is represented by Formula 1:

$$( A ) m - R - ( B ) n ; \qquad ( Formula 1 )$$

where “R” is an organic segment;
where “A” is a first terminal functional group comprising a protected amine, such as, but not limited to, benzyloxycarbonyl (Cbz)-protected amine or a tert-butoxycarbonyl (Boc)-protected amine;
where “B” is a second terminal functional group comprising at least one of an isocyanate and a chloroformate ester;
where “m” is 1; and
where “n” is 1, 2, or 3.

The above and other aspects, embodiments, features and benefits of the present disclosure will be readily apparent from the following detailed description.

### BRIEF DESCRIPTION OF THE DRAWINGS

FIGS. 1A-1E schematically illustrate an example process for forming a hydrolytically stable iodinated multi-functional compound, in accordance with an embodiment of the present disclosure.

FIGS. 2A-2E schematically illustrate another example process for forming a hydrolytically stable iodinated multi-functional compound, in accordance with an embodiment of the present disclosure;

FIGS. 3A-3C schematically illustrate another example process for forming hydrolytically stable iodinated multi-functional compound, in accordance with an embodiment of the present disclosure;

FIGS. 4A-4C schematically illustrate another example process for forming hydrolytically stable iodinated multi-functional compound, in accordance with an embodiment of the present disclosure;

FIGS. 5A-5B schematically illustrate another example process for forming hydrolytically stable iodinated multi-functional compound, in accordance with an embodiment of the present disclosure;

FIG. 6 schematically illustrates a delivery device, in accordance with an embodiment of the present disclosure; and

FIG. 7 schematically illustrates a delivery device, in accordance with another embodiment of the present disclosure.

### DETAILED DESCRIPTION

In some aspects, the present disclosure provides a hydrolytically stable iodinated multi-functional compound comprising: a multi-functional compound having functional groups that are reactive with a reactive group of a multi-arm polymer; an iodinated polyol having reactive moieties including two or more hydroxyl groups, and one or more iodinated aromatic groups; and a linker between a reactive moiety of the iodinated polyol and a functional group of the multi-functional compound, wherein the linker includes a carbamate linkage or a carbonate linkage

In some embodiments, a linker between a reactive moiety of the iodinated polyol and a functional group of the multi-functional compound in an iodinated hydrogel network has a hydrolytic stability that is greater than the hydrolytic stability of ester linkages that are also present in the iodinated hydrogel network. For instance, the linker can have a hydrolytic stability in the presence of water that is greater than a hydrolytic stability of a comparative esters linkage in the presence of water. For example, in some embodiments, the moieties comprise a linker, which may be selected from the following groups, among others: a carbonate linkage,

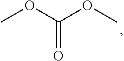

and a carbamate linkage,

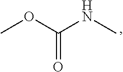

In some embodiments, the linker is manifested as a carbonate linkage. For instance, each of the heterobifunctional linkers e.g., within a hydrolytically stable iodinated multi-functional compound may be a carbonate linkage. However, in some embodiments the linker is manifested as carbamate linkage. For instance, each of the heterobifunctional linkers e.g., within the hydrolytically stable iodinated multi-functional compound may be a carbamate linkage.

In some embodiments, the linkers which are coupled to the iodine-containing compound and the multi-functional can be manifested as heteropolyfunctional linkers having different function groups (e.g., different terminal functional groups). For instance, the heteropolyfunctional linkers can be manifested as heterobifunctional linkers. The heterobifunctional linkers can include a first reactive moiety or first functional group (e.g., a first terminal functional group) that can form hydrolytically stable covalent bonds with various iodinated polymers (e.g., iodixanol, etc.), as described herein. The first functional group can include, but are not limited to, an isocyanate or chloroformate ester. For instance, in some embodiments the first functional group is an isocyanate. In such instances, a carbamate linker can be formed, as described herein. Yet, in some embodiments the first functional group is a chloroformate ester. In such instances, a carbonate linker can be formed, as described herein.

In some embodiments, the heteropolyfunctional linker is tert-butyl N-(2-Isocyanatoethyl)carbamate (CAS #284049-22-5) or tert-butyl N-(3-isocyanatopropyl)carbamate (CAS #76197-73-4).

In some embodiments, the heteropolyfunctional linker is a linear heterobifunctional linker. For instance, in some embodiments, the linear heterobifunctional linker is a protected amino chloroformate. Examples of suitable protected amino chloroformates include 4-(t-butoxycarbonylamino)butyl carbonochloridate, 2-(benzyloxycarbonylamino)ethyl carbonochloridate, 2-[2-(tbutoxycarbonylamino)ethyldisulfanyl]ethyl carbonochloridate, 3-(t-butoxycarbonylamino)propyl carbonochloridate, 2-[2-(tbutoxycarbonylamino) ethoxy]ethyl carbonochloridate, 6-(9H-fluoren-9-ylmethoxycarbonylamino)hexyl carbonochloridate, 2-(9H-fluoren-9-ylmethoxycarbonylamino)ethyl carbonochloridate, and chlorocarbonyl 2-(tritylamino)acetate.

As mentioned, the heterobifunctional linkers (or heteropolyfunctional hydrolysable linkers) also include a second functional group (e.g., an amine) that can be coupled to the multi-functional crosslinker (e.g., trilysine). For instance, the second functional group can be an amine such as a protected amine that can be deprotected (e.g., under mild acidic conditions) subsequent to formation of a bond between the first functional group and the iodinated polyol (e.g., a hydroxyl group thereof). For instance, the protected amine can include, but is not limited to, a benzyloxycarbonyl (Cbz)-protected amine or a tert-butoxycarbonyl (Boc)-protected amine. The deprotected second functional group (e.g., an amine) can subsequently be coupled to a corresponding functional group in the multi-functional compound.

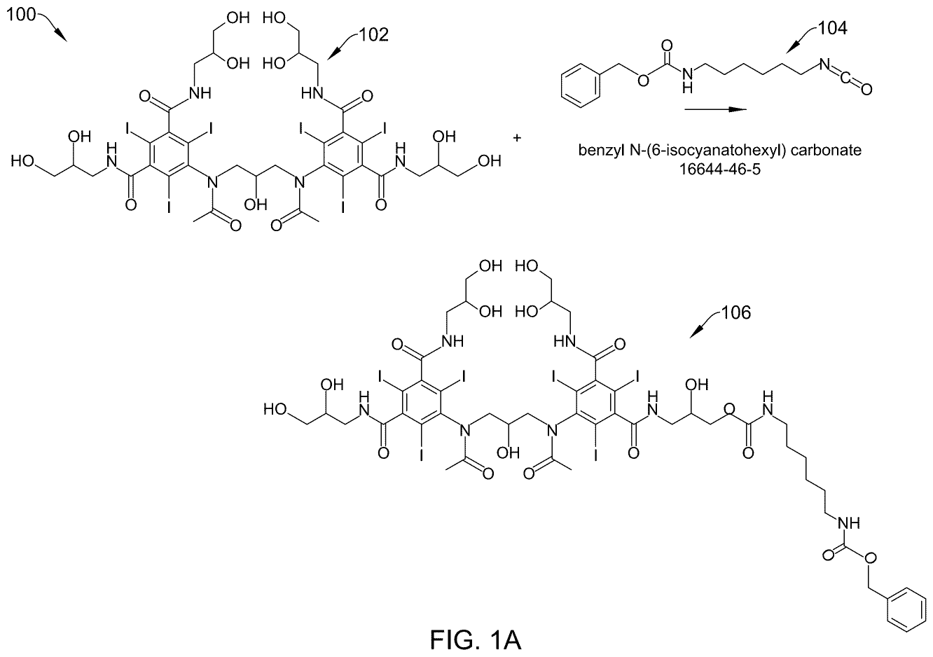

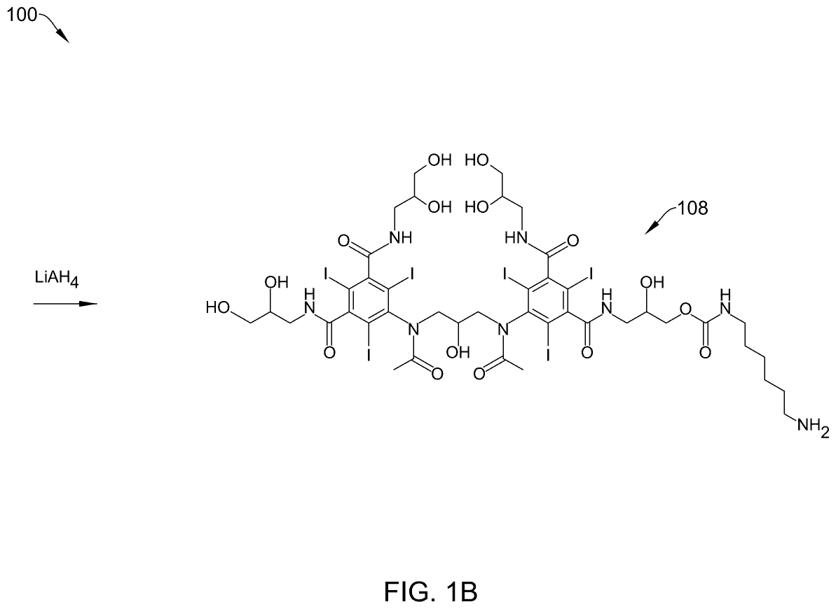

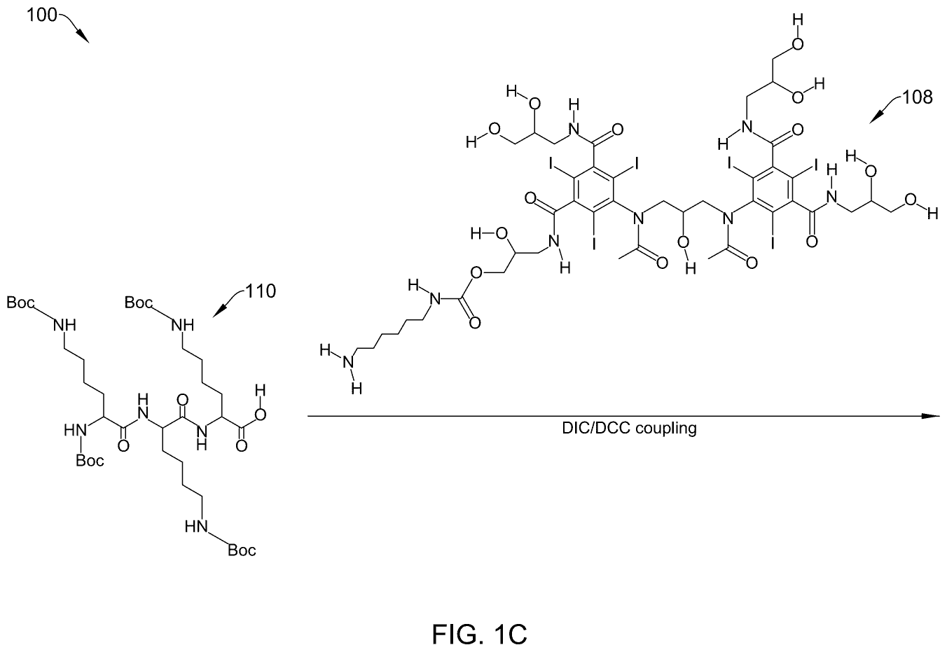

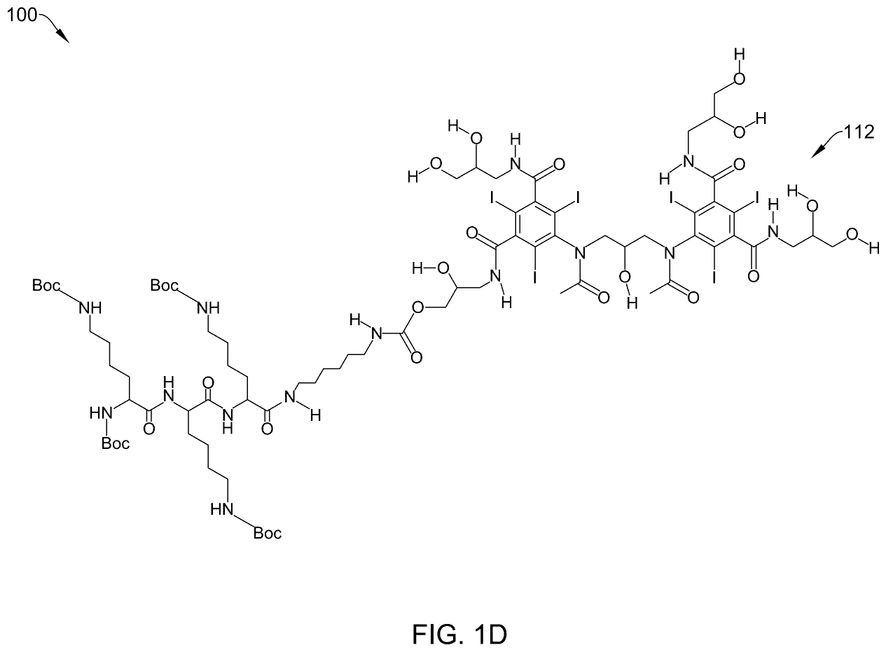

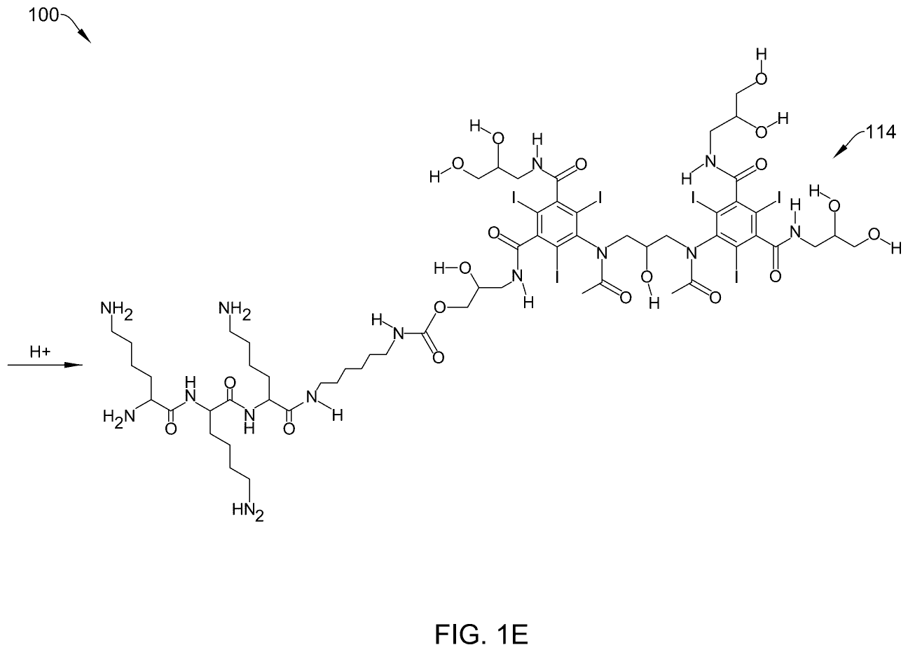

FIGS. 1A-1E schematically illustrate an example process 100 for forming a hydrolytically stable iodinated multi-functional compound, in accordance with an embodiment of the present disclosure. As illustrated in FIGS. 1A-1E, an iodinated polyol in the form of iodixanol (as illustrated at 102 in FIG. 1A) can be coupled to peptides through the use of a linker (e.g., a heterobifunctional linker), containing at least one isocyanate, and one orthogonally protected group, such as a Cbz-protected amine, such as benzyl N-(6-isocyanatohexyl)carbamate (CAS #16644-46-5) (as illustrated at 104 in FIG. 1A) which reacts with the iodixanol (102) to form an intermediate product (as illustrated at 106 in FIG. 1A) including a carbamate linkage and a Cbz-protected amine. Once formed, the Cbz-protected amine in the intermediate product can be deprotected with, for example, hydrochloric acid, 1 to yield [3-[[3-[acetyl-[3-[N-acetyl-3,5-bis(2,3-dihydroxypropylcarbamoyl)-2,4,6-triiodoanilino]-2-hydroxy-propyl]amino]-5-(2,3dihydroxypropylcarbamoyl)-2,4,6-triiodo-benzoyl]amino]-2-hydroxy-propyl]N-(6-aminohexyl)carbamate (as illustrated at 108 in FIG. 1B). The [3-[[3-[acetyl-[3-[N-acetyl-3,5-bis(2,3-dihydroxypropylcarbamoyl)-2,4,6-triiodoanilino]-2-hydroxy-propyl]amino]-5-(2,3dihydroxypropylcarbamoyl)-2,4,6-triiodo-benzoyl]amino]-2-hydroxy-propyl]N-(6-aminohexyl)carbamate (108) can subsequently be reacted with (e.g., undergo DIC or DCC coupling with) a multi-functional compound in the form of trilysine (as illustrated at 110 in FIG. 1C) e.g., reacted with a carboxylic acid of trilysine to yield an intermediate hydrolytically stable iodinated multi-functional compound (as illustrated at 112 in FIG. 1D) that can subsequently undergo mild acidification to yield the final hydrolytically stable iodinated multi-functional compound (as illustrated at 114 in FIG. 1E) which includes a carbamate linkage between the residue of the iodinated polyol and the residue of the multi-functional compound. As mentioned, the carbamate linkage can yield enhanced hydrolytic stability of the final hydrolytically stable iodinated multi-functional compound (114), as compared to other types of linkages such as ester linkages.

FIGS. 1A-1E, illustrate the use of a particular linker (e.g., a heterobifunctional linker), containing at least one isocyanate, and one orthogonally protected amine, such as a Cbz-protected amine in the form of benzyl N-(6-isocyanatohexyl)carbamate (CAS #16644-46-5). However, as detailed herein the use of alternate linkers, iodinated polyols, and/or other components is possible. For instance, in some embodiments, 1,1-Dimethylethyl N-(2-isocyanatoethyl)carbamate (CAS 284049-22-5) may be employed with an iodinated polyol and a multi-functional compound using the methods described herein (e.g., protection, deprotection, and/or acidification) to yield a final hydrolytically stable iodinated multi-functional compound which includes a carbamate linkage between the residue of the iodinated polyol and the residue of the multi-functional compound.

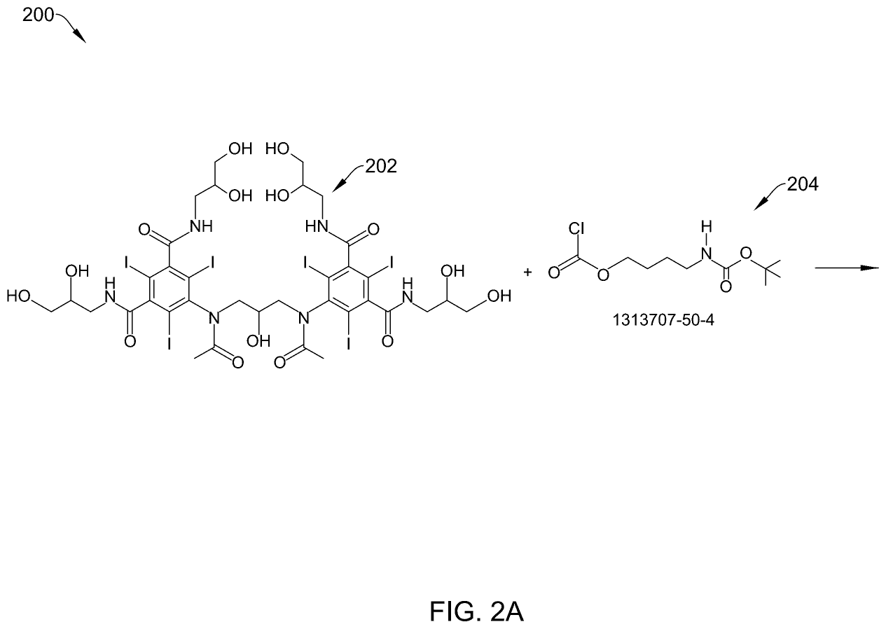

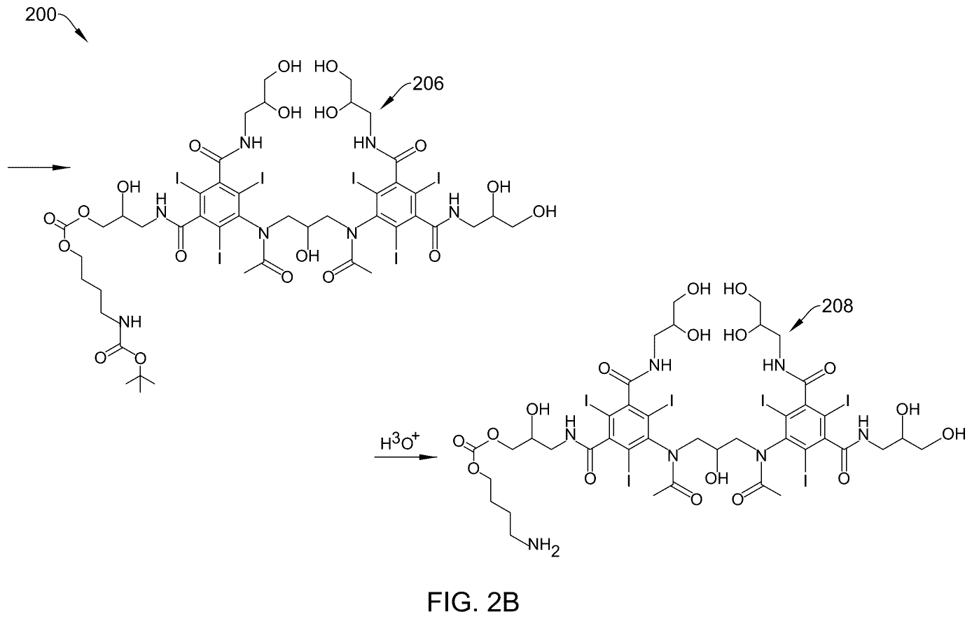

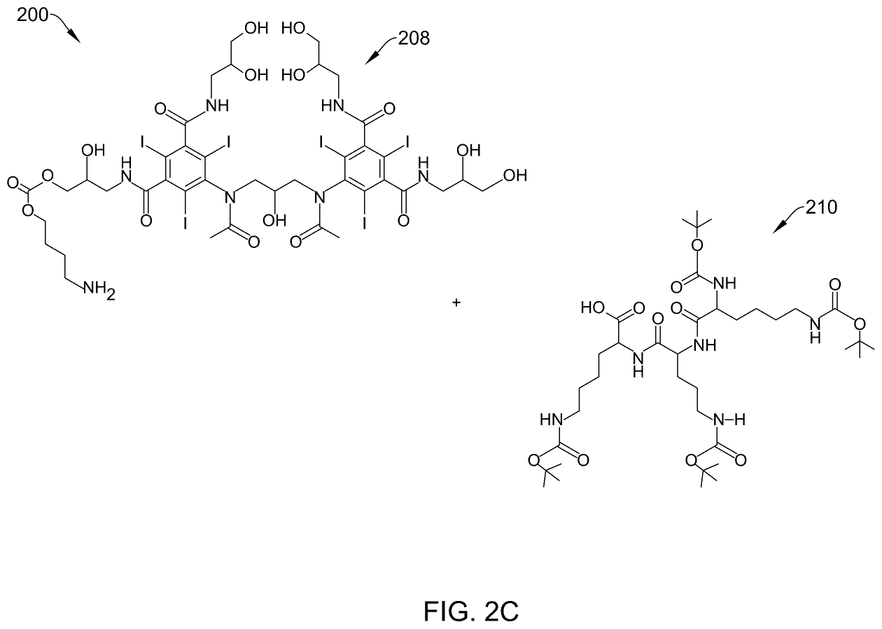

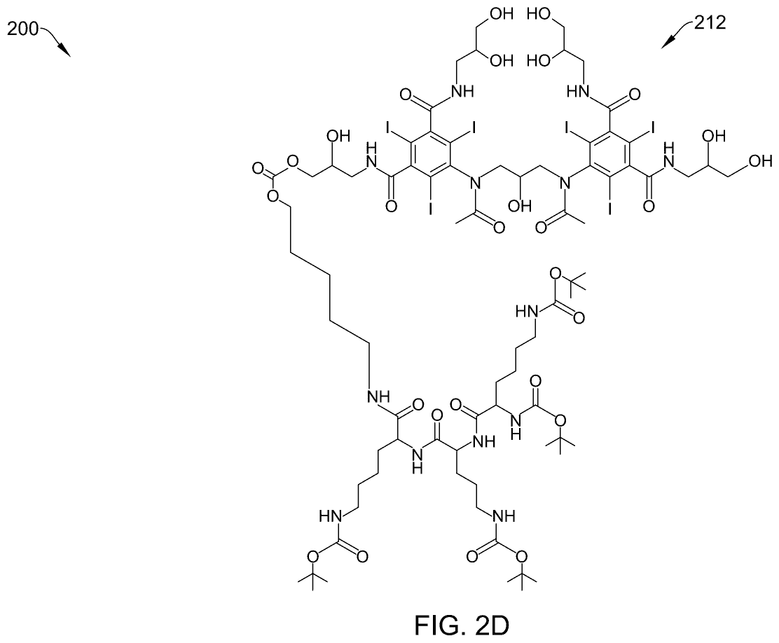

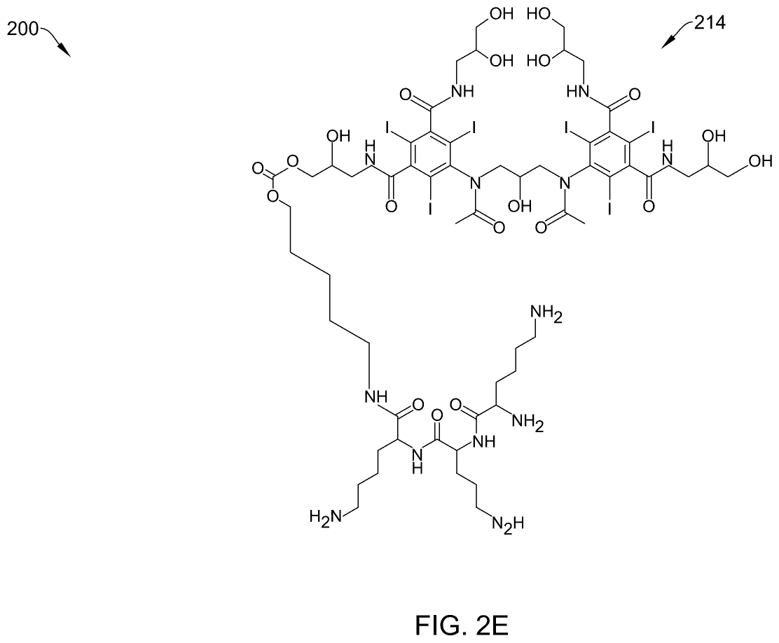

FIGS. 2A-2E schematically illustrate another example process 200 for forming a hydrolytically stable iodinated multi-functional compound, in accordance with an embodiment of the present disclosure. As illustrated in FIGS. 2A-2E an iodinated polyol in the form of iodixanol (as illustrated at 202 in FIG. 2A) can be coupled to peptides through the use of a linker (e.g., a heterobifunctional linker), containing at least one chloroformate ester, and one orthogonally protected group, such as a Cbz-protected amine, such as benzyl N-(6-isocyanatohexyl)carbamate (CAS #16644-46-5) (as illustrated at 204 in FIG. 2A) which reacts with an alcohol group in the iodixanol (202) to form an intermediate product (as illustrated at 206 in FIG. 2B) including a carbonate linkage and a t-boc protected amine. Once formed, the t-boc protected amine in the intermediate product (206) can be deprotected using, for example, hydrochloride acid, to yield [3-[[3-[acetyl-[3-[N-acetyl-3,5-bis(2,3-dihydroxypropylcarbamoyl)-2,4,6-triiodoanilino]-2-hydroxy-propyl]amino]-5-(2,3-dihydroxypropylcarbamoyl)-2,4,6-triiodo-benzoyl]amino]-2-hydroxy-propyl]N-(6-aminohexyl)carbamate, (as illustrated at 208), a water soluble iodinated species with improved hydrolytic stability. [3-[[3-[acetyl-[3-[N-acetyl-3,5-bis(2,3-dihydroxypropylcarbamoyl)-2,4,6-triiodoanilino]-2-hydroxy-propyl]amino]-5-(2,3-dihydroxypropylcarbamoyl)-2,4,6-triiodo-benzoyl]amino]-2-hydroxy-propyl]N-(6-aminohexyl)carbamate (208 in FIG. 2B) can subsequently be reacted with (e.g., undergo DIC or DCC coupling with) a multi-functional compound in the form of trilysine (as illustrated at 210 in FIG. 2C) e.g., reacted with a carboxylic acid of the trilysine to yield an intermediate hydrolytically stable iodinated multi-functional compound (as illustrated at 212 in FIG. 2D) that can subsequently undergo mild acidification to yield the final hydrolytically stable iodinated multi-functional compound (as illustrated at 214 in FIG. 2E) which includes a carbonate linkage between the residue of the iodinated polyol and the residue of the multi-functional compound. As mentioned, the carbonate linkage can yield enhanced hydrolytic stability of the final hydrolytically stable iodinated multi-functional compound (214), as compared to other types of linkages such as ester linkages.

While FIGS. 1A-1E and 2A-2E employ particular examples of iodinated polyols, multi-functional compounds, and linkers, other types of iodinated polyols, multi-functional compounds, and linkers such as those described herein can be employed. In some embodiments, the linkage is a residue of a heteropolyfunctional linker. In some embodiments, the linkers are linked between a hydroxyl group of the iodinated polyol and a functional group of the functional groups of the multi-functional compound. In some embodiments, the linker is a residue of a heteropolyfunctional linker and is linked between a hydroxyl group of the iodinated polyol and a functional group of the functional groups of the multi-functional compound. In some embodiments, the heteropolyfunctional linker can be represented by the Formula 1:

$$( A ) m - R - ( B ) n ; \qquad ( Formula 1 )$$

where “R” is an organic segment;
where “A” is a first terminal functional group comprising a protected amine such as a benzyloxycarbonyl (Cbz)-protected amine or a tert-butoxycarbonyl (Boc)-protected amine;
where “B” is a second terminal functional group comprising at least one of an isocyanate and a chloroformate ester;
where “m” is 1; and
where “n” is 1, 2, or 3

Specific examples of suitable organic segments include, but are not limited to, alkyl chains, ether containing species, amide containing species, amine containing species, alcohol containing species, segments including carbon, oxygen, and/or nitrogen atoms.

Specific examples of suitable linkers include, but are not limited to, those selected from: 4-(t-butoxycarbonylamino)butyl carbonochloridate (CAS #1313707-50-4),

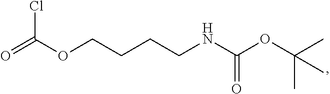 [SMILES: CC(C)(C)OC(=O)NCCCCOC(=O)Cl]

2-(benzyloxycarbonylamino)ethyl

### Carbonochloridate (CAS #77987-53-2),

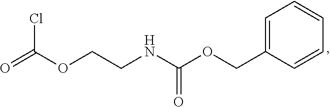 [SMILES: O=C(Cl)OCCNC(=O)OCc1ccccc1]

2-[2-(tbutoxycarbonylamino) ethyldisulfanyl]ethyl carbonochloridate (CAS #877865-47-9),

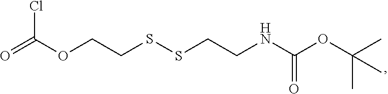 [SMILES: CC(C)(C)OC(=O)NCCSSCCOC(=O)Cl]

3-(t-butoxycarbonylamino)propyl carbonochloridate (CAS #1313707-49-1),

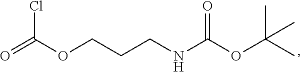

2-[2-(tbutoxycarbonylamino) ethoxy]ethyl carbonochloridate (CAS #1710364-91-2),

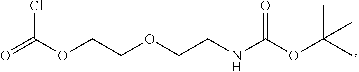 [SMILES: CC(C)(C)OC(=O)NCCOCCOC(=O)Cl]

6-(9H-fluoren-9-ylmethoxycarbonylamino)hexylcarbonochloridate (CAS #149151-38-2),

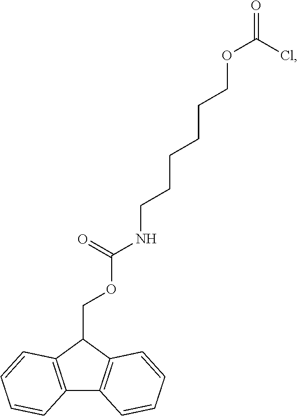 [SMILES: O=C(Cl)OCCCCCCNC(=O)OCC1c2ccccc2-c2ccccc21]

2-(9H-fluoren-9-ylmethoxycarbonylamino)ethylcarbonochloridate (CAS #1037509-28-6),

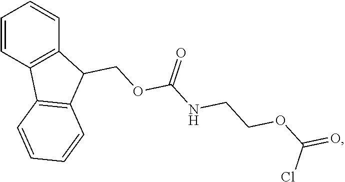 [SMILES: O=C(Cl)OCCNC(=O)OCC1c2ccccc2-c2ccccc21]

and
chlorocarbonyl 2-(tritylamino)acetate (CAS #858254-61-2),

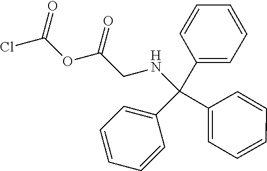 [SMILES: O=C(Cl)OC(=O)CNC(c1ccccc1)(c1ccccc1)c1ccccc1]

In some embodiments, the linkers can be manifested as branched heteropolyfunctional linkers. The presence of branched heteropolyfunctional linkers permits multiple iodinated functional groups to be added (e.g., at the same time). In such instances, the branched heteropolyfunctional linkers can have one protected amine (e.g., a benzyloxycarbonyl (Cbz)-protected amine or a tert-butoxycarbonyl (Boc)-protected amine) and two or more chloroformate esters. The presence of two or more chloroformate esters permits the quantity of iodinated functional groups to be increased beyond one iodinated species per linker (e.g., to two more iodinated functional groups per branched heteropolyfunctional linker). Examples of suitable branched heteropolyfunctional linkers include, but are not limited to:

2-[benzyloxycarbonyl(2-chlorocarbonyloxyethyl)amino]ethyl carbonochloridate (CAS #167899-32-3),

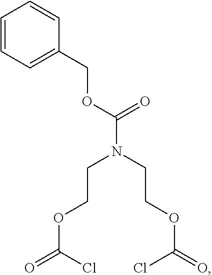 [SMILES: O=C(Cl)OCCN(CCOC(=O)Cl)C(=O)OCc1ccccc1]

and 6-[2-[benzyloxycarbonyl-[2-[6-chlorocarbonyloxyhexyl(methyl)carbamoyl]oxyethyl]amino]ethoxycarbonylmethyl-amino]hexyl carbonochloridate (CAS #167899-34-5).

(CAS #167899-34-5),

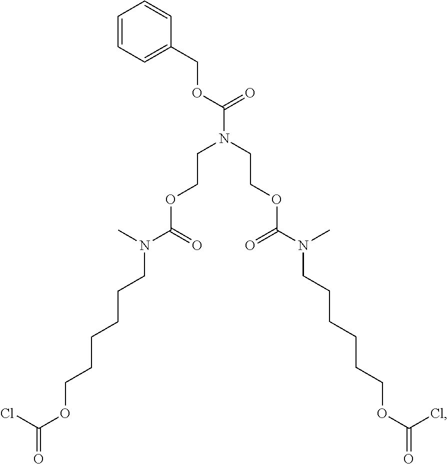 [SMILES: CN(CCCCCCOC(=O)Cl)C(=O)OCCN(CCOC(=O)N(C)CCCCCCOC(=O)Cl)C(=O)OCc1ccccc1]

among other suitable compounds such as those described herein.

In addition to expanding the number of iodinated groups that can be added, the branched polyfunctional linkers also permit coupling to various other types of iodinated species (e.g., other than those listed in Table 1).

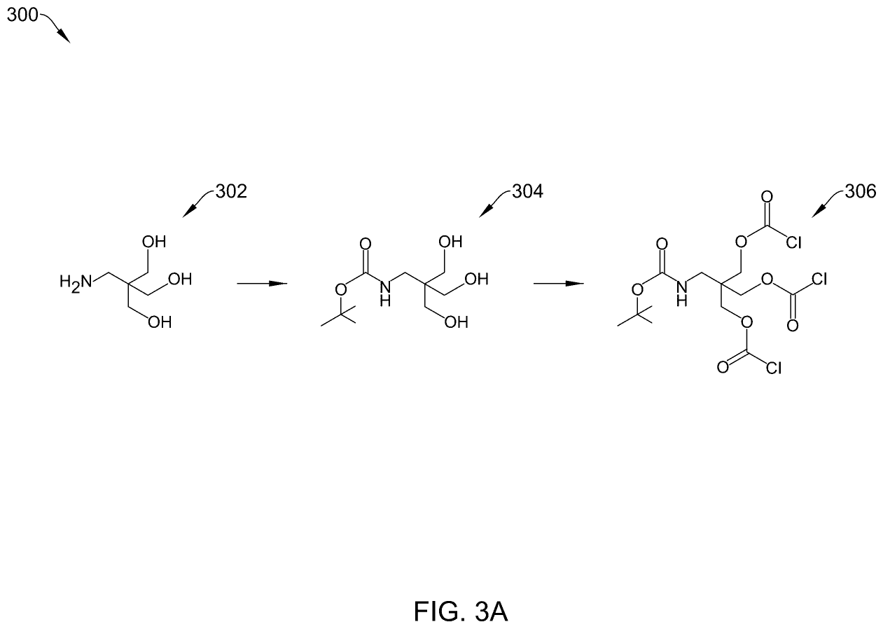

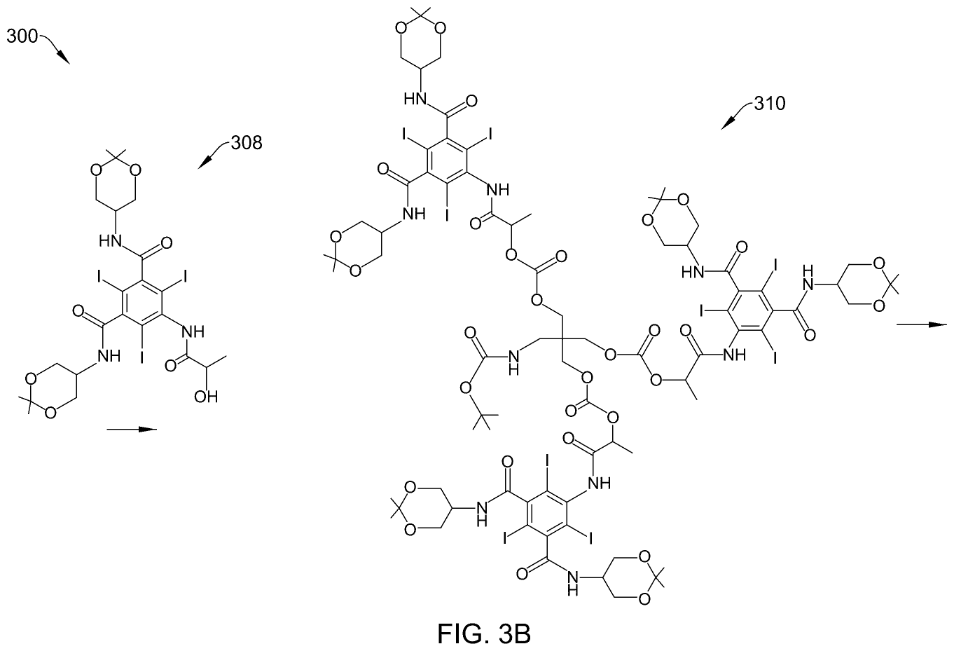

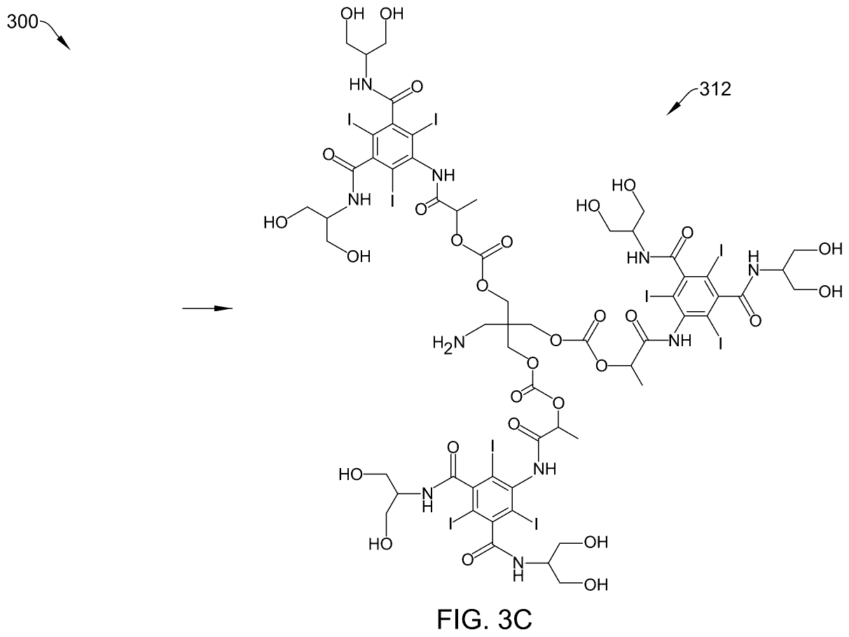

For instance, FIGS. 3A-3C schematically illustrate another example process 300 for forming hydrolytically stable iodinated multi-functional compound, in accordance with an embodiment of the present disclosure. As illustrated in FIGS. 3A-3C, the amine group in 2-(aminomethyl)-2-(hydroxymethyl)propane-1,3-diol (as illustrated at 302 in FIG. 3A) can be protected with, for example, a t-Boc group (as illustrated at 304 in FIG. 3A), and then converted to the trichloroformate ester (as illustrated at 306 in FIG. 3A). The trichloroformate ester (a branched polyfunctional linker) can be reacted with a protected derivative of iopamidol (as illustrated at 308 in FIG. 3B) to yield an intermediate for forming a hydrolytically stable iodinated multi-functional compound (as illustrated at 310 in FIG. 3C). Upon deprotection (e.g., under mild acidic conditions) as indicated, the final intermediate for forming a hydrolytically stable iodinated multi-functional compound (as indicated at 312 in FIG. 3C) is generated, which includes nine total iodine atoms being added to each crosslink site. One or more protected amines in the final intermediate for forming a hydrolytically stable iodinated multi-functional compound (312) can be deprotected and coupled to a multi-functional compound such as trilysine via the same or similar mechanisms as described with respect to FIGS. 1A-1E. and/or FIGS. 2A-E to yield a hydrolytically stable iodinated multi-functional compound formed form a residue of the multi-functional compound and a residue of the final intermediate for forming a hydrolytically stable iodinated multi-functional compound (312).

While FIGS. 3A-3C employ a protected derivative of iopamidol, it is noted that a similar approach can be taken with various other iodinated polyols. For instance, a similar approach can be undertaken with a protected derivative of iodixanol, which would then result in 18 iodine atoms per functional group in the final hydrolytically stable iodinated multi-functional compound obtained therefrom.

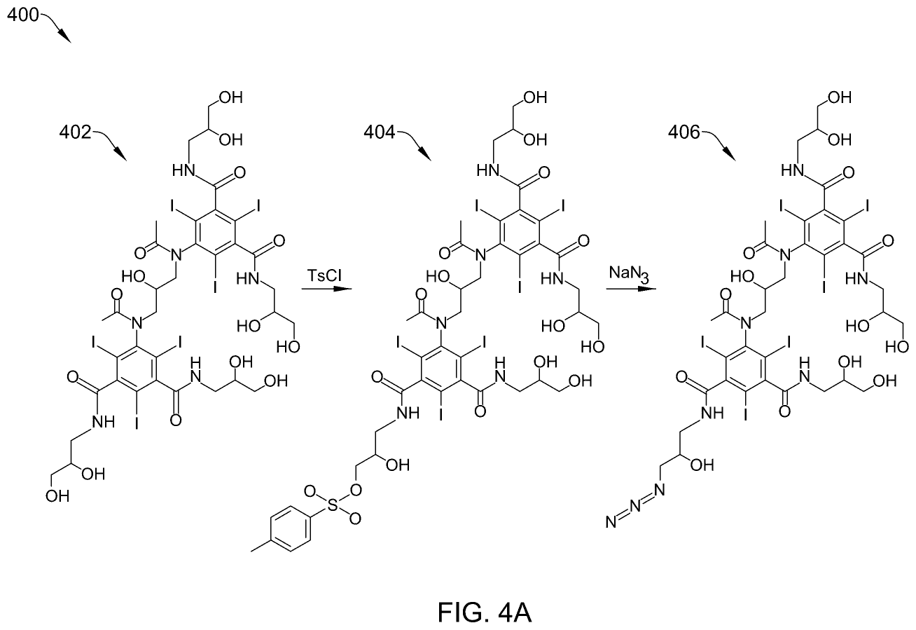

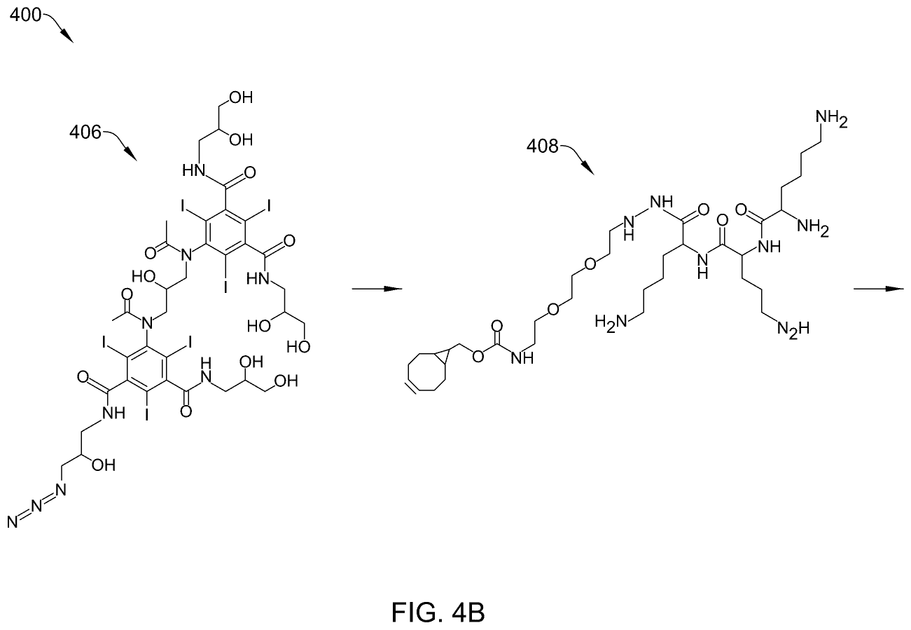

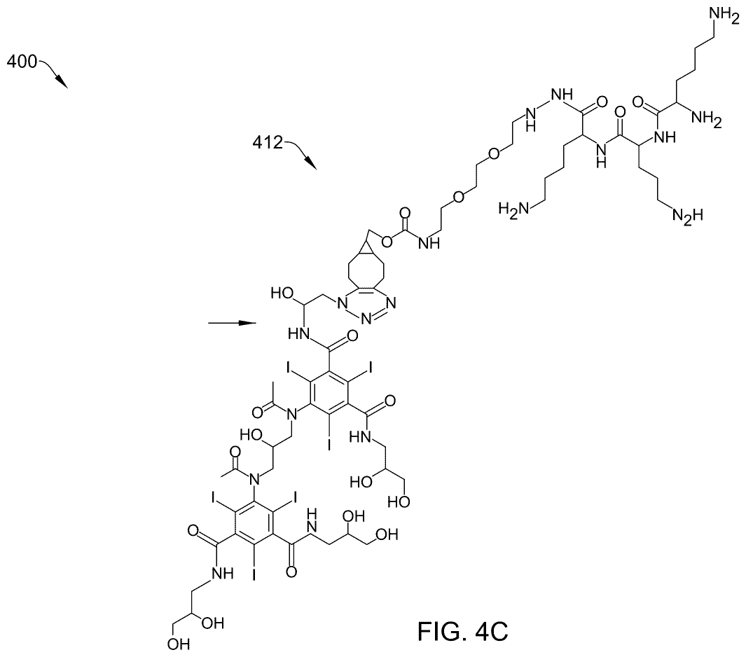

In some embodiments, click reactive chemistries can be employed to promote aspects herein. For instance in some embodiments, the iodinated polyols herein can be manifested as azide-functionalized iodinated polyols that include an azide functional group. In such instances, the linker herein can be formed between the azide functional group and a corresponding functional group of the multi-functional compound. For instance, FIGS. 4A-4C schematically illustrate another example process 400 for forming hydrolytically stable iodinated multi-functional compound, in accordance with an embodiment of the present disclosure. As illustrated in FIGS. 4A-4C, a corresponding click-reactive trilysine derivative (as illustrated at 412 in FIG. 4C) can be made, by coupling t-boc protected trilysine (as illustrated at 408 in FIG. 4B) with a commercially available strained amino-alkyne, such as 9-bicyclo[6.1.0]non-4-ynylmethyl N-[2-[2-(2-aminoethoxy)ethoxy]ethyl]carbamate (CAS #1263166-93-3) (as illustrated at 408 in FIG. 4B) using standard peptide coupling strategies. The iodixanol (402 as illustrated in FIG. 4A) can be reacted with p-toluenesulfonyl chloride and subsequently can be reacted with sodium azide (NaN₃) (as illustrated at 404 in FIG. 4A) to convert at least one hydroxyl group into a click-reactive group such as an azide, thereby forming the click-reactive compound (as illustrated at 406 in FIG. 4A). The click-reactive compound (406) can be reacted with a multi-functional compound such as the t-boc protected trilysine (408) to form a hydrolytically stable iodinated multi-functional compound (e.g., a click-reactive trilysine derivative, as illustrated at 412 in FIG. 4C).

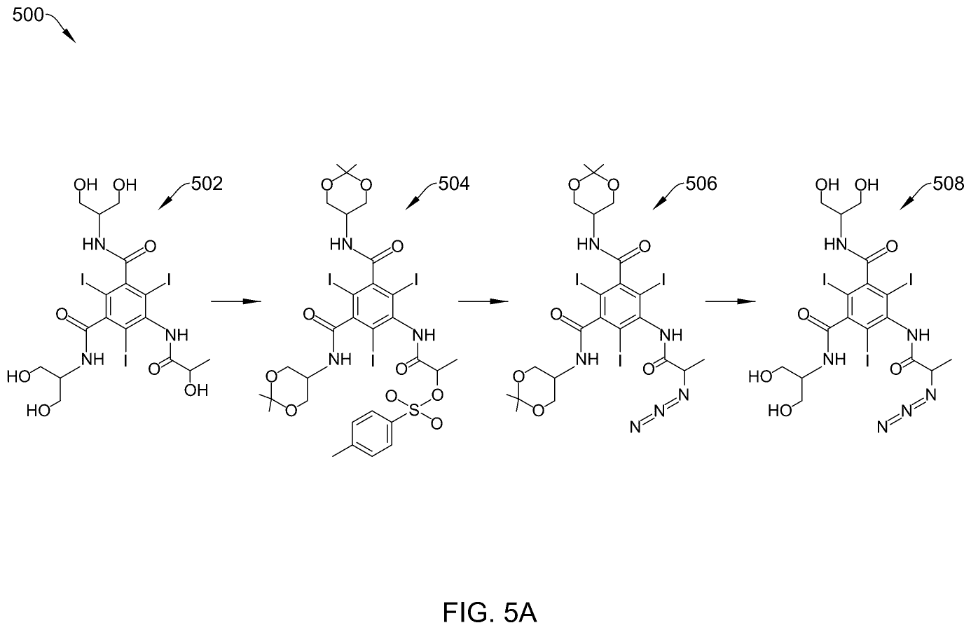

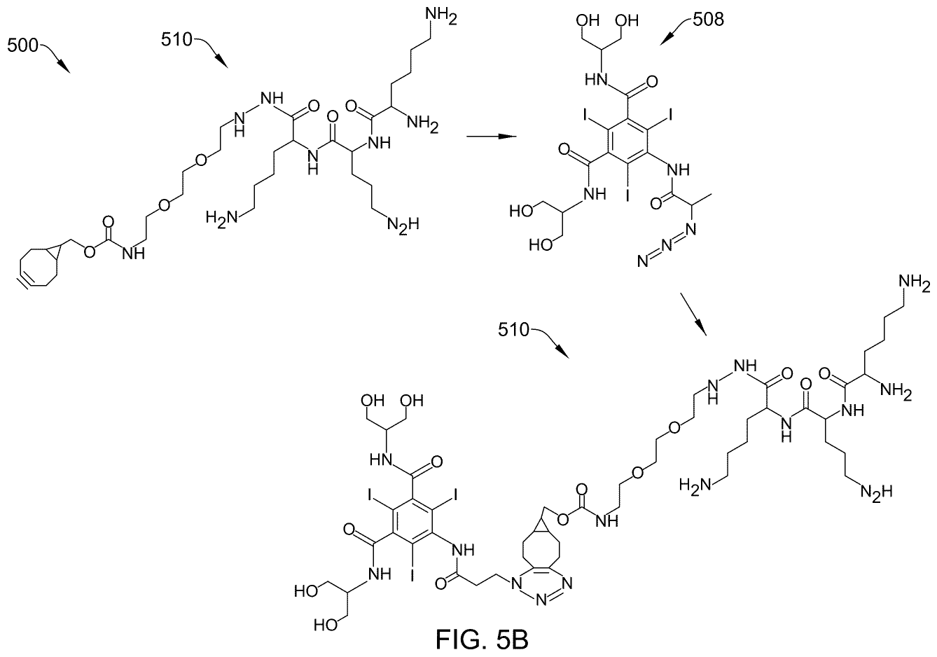

Similar to the FIGS. 4A-4C, in some embodiments a click-reactive group can be formed from an individual alcohol group of a pair of alcohol groups of an iodinated polyol. For example, FIGS. 5A-5B schematically illustrates another example process 500 for forming hydrolytically stable iodinated multi-functional compound, in accordance with an embodiment of the present disclosure. As illustrated in FIGS. 5A-5B, an iodinated polyol such as iopamidol (as illustrated at 502 in FIG. 5A), where paired alcohols are protected, can undergo conversion of one of the protected alcohols. For instance, one of the paired alcohol groups of iopamidol (502) can be protected (e.g., t-boc protected, Cbz protected, or FMOC protected), (as illustrated at 504 in FIG. 5A) and can other alcohol group can be reacted with p-toluenesulfonyl chloride and subsequently can be reacted with sodium azide (NaN₃) to convert at least one hydroxyl group into a click-reactive group such as an azide, as illustrated at 506. The protected alcohol group in the compound 506 can then be deprotected to yield the click-reactive compound (as illustrated at 508 in FIG. 5B). While not expressly shown in FIG. 5B, the click-reactive compound (508) can be reacted with a multi-functional compound such as the t-boc protected trilysine to form a hydrolytically stable iodinated multi-functional compound (e.g., a click-reactive trilysine derivative), illustrated at 510 in FIG. 5B.

Thus, it is noted that the systems and methods herein can include traditional coupling (e.g., as described with respect to FIGS. 1A-1E, 2A-2E, and 3A-3C) to form hydrolytically stable iodinated multi-functional compounds or can employ alternate coupling methods (e.g., via click reactions as described with respect to FIGS. 4A-4C and 5A-5B) to form hydrolytically stable iodinated multi-functional compounds.

Reactive multi-arm polymers in accordance with the present disclosure include polymers that comprise a plurality of polymer arms linked to a core region. The reactive multi-arm polymers may have two, three, four, five, six, seven, eight, nine, ten, eleven, twelve, thirteen, fourteen, fifteen, sixteen, seventeen, eighteen, nineteen, twenty, twenty-five, thirty or more polymer arms.

As discussed in more detail below, in some embodiments, the core region comprises a residue of a multi-functional initiator, specifically, a residue of an polyol initiator having two, three, four, five, six, seven, eight, nine, ten, eleven, twelve, thirteen, fourteen, fifteen, sixteen, seventeen, eighteen, nineteen, twenty, twenty-five, thirty or more hydroxyl groups, with the number of polymer arms corresponding to the number of functional groups, specifically, the number of hydroxyl groups, in the initiator that is used to form the reactive multi-arm polymer.

The polymer arms of the reactive multi-arm polymers may each comprise a polymer segment linked to the core region and a terminal reactive moiety.

Reactive moieties may be selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups, among others.

Electrophilic groups include cyclic imide ester groups, including succinimide ester groups,

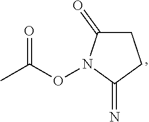

maleimide ester groups, glutarimide ester groups, diglycolimide ester groups, phthalimide ester groups, and bicyclo[2.2.1]hept-5-ene-2,3-dicarboxylic acid imide ester groups,

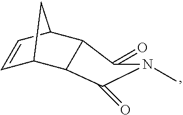

imidazole ester groups, imidazole carboxylate groups and benzotriazole ester groups, among other possibilities. Nucleophilic groups include primary amine groups, thiol groups and hydroxyl groups, among other possibilities. Diene containing groups include furan groups and tetrazine groups. Dienophile groups include norbornene groups and maleimide groups. Alkenyl-containing groups include vinyl groups, acryloyl groups, methacryloyl groups and strained alkene groups, for example, from cyclooct-4-en-1-yl groups,

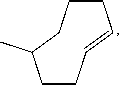

among other possibilities. Strained alkyne groups include (1R,8S,9s)-bicyclo[6.1.0]non-4-yn-9-yl groups,

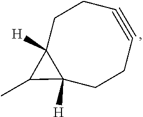

among other possibilities.

Polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(O-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the reactive multi-arm polymers of the present disclosure typically contain between 10 and 1000 monomer units or more.

As previously noted, in various embodiments, the reactive multi-arm polymers of the present disclosure have two or more polymer arms that extend from a core region. In some of these embodiments, the core region comprises a residue of a polyol comprising two or more hydroxyl groups, which is used to form the polymer arms. In certain beneficial embodiments, the core region comprises a residue of a polyol that contains two, three, four, five, six, seven, eight, nine, ten, eleven, twelve, thirteen, fourteen, fifteen, sixteen, seventeen, eighteen, nineteen, twenty, twenty-five, thirty, or more hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, polyhedral oligomeric silsesquioxanes (POSS), catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In some embodiments, iodinated polyols may be employed to provide the resulting multi-arm polymer with radiopacity. In some of these embodiments, the iodinated polyols are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others. Specific examples of iodinated polyols for use in the present disclosure include known iodinated contrast agents, whose biocompatibility has been demonstrated to be reasonably well tolerated, which include iopromide [CAS: 73334-07-3], 5-Acetamido-N,N′-bis(2,3-dihydroxypropyl)-2,4,6-triiodoisophthalamide [CAS: 31127-80-7], Metrizamide [31112-62-6], 1,3-Benzenedicarboxamide, 5-(acetylamino)-N1,N3-bis(2,3-dihydroxypropyl)-2,4,6-triiodo-N1-methyl- (ACI) [CAS: 76350-28-2], iopamidol [CAS: 60166-93-0], Iomeprol [CAS: 78649-41-9], 1,3-Benzenedicarboxamide, N1,N3-bis[2-hydroxy-1-(hydroxymethyl)ethyl]-5-[(2-hydroxy-1-oxopropyl)amino]-2,4,6-triiodo- (ACI) [CAS: 0208-45-9], ioxilan [CAS: 107793-72-6], iopentol [CAS: 89797-00-2], ioversol [87771-40-2], iohexol [CAS: 66108-95-0], iobitridol [136949-58-1], iodixanol [CAS: 92339-11-2], and [1,3-Benzenedicarboxamide, 5,5′-[(1,3-dioxo-1,3-propanediyl)bis(methylimino)]bis[N,N′-bis[2,3-dihydroxy-1-(hydroxymethyl)propyl]-2,4,6-triiodo- (9CI, ACI)][CAS: 79770-24-4].

In particular embodiments where an ester is selected as a hydrolysable linkage between the multi-arm polymer and the multi-functional compound, terminal hydroxyl groups of the polymer segments maybe reacted with acyclic anhydride compound (e.g., glutaric anhydride, succinic anhydride, malonic anhydride, adipic anhydride, diglycolic anhydride, 1,3-acetonedicarboxylic acid anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, a 1,3-acetonedicarboxylic-acid-end-capped segment, and so forth.

The preceding cyclic anhydrides, among others, may be reacted with a hydroxy-terminated multi-arm hydrophilic precursor polymer under basic conditions to form a carboxylic-acid-terminated precursor polymer comprising a carboxylic acid end group that is linked to a polymer segment through a hydrolysable ester group.

A reactive moiety may then be linked to the carboxylic-acid-terminated precursor polymer. For example, in particular embodiments where a cyclic imide ester group is employed as a reactive group, an N-hydroxy cyclic imide compound (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester group (e.g., a succinimide ester group, a maleimide ester group, a glutarimide ester group, a phthalimide ester group, a bicyclo[2.2.1]hept-5-ene-2,3-dicarboxylic acid imide ester group, etc.) that is linked to a polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, succinimidyl diglycolate groups, and succinimidyl 1,3-acetonedicarboxylate groups (1,3-acetonedicarboxylate groups may also be referred to herein as 3-oxopentanedioate groups), among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, bicyclo[2.2.1]hept-5-ene-2,3-dicarboxylic acid imidyl diglycolate groups, and bicyclo[2.2.1]hept-5-ene-2,3-dicarboxylic acid imidyl 1,3-acetonedicarboxylate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, maleimidyl diglycolate groups, and maleimidyl 1,3-acetonedicarboxylate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, and glutarimidyl 1,3-acetonedicarboxylate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, phthalimidyl diglycolate groups, and phthalimidyl 1,3-acetonedicarboxylate groups, among others.

Using various suitable chemistries, the moieties that comprise heterobifunctional linkers other than the linkers described above can be employed to couple an iodinated polyol to a multi-functional compound (e.g., crosslinker). Such linkers have a hydrolytic stability that is greater than the hydrolysable ester linkages and may be selected, for example, from a carbonate linkage and a carbamate linkage.

In some aspects, the present disclosure provides radiopaque hydrogels that comprise a crosslinked reaction product of (a) a reactive multi-arm polymer as described herein and (b) a hydrolytically stable iodinated multi-functional crosslinking compound having a plurality of complementary reactive moieties that is reactive with the reactive moieties of the reactive multi-arm polymer.

In some aspects, the present disclosure provides radiopaque hydrogels that comprise a crosslinked reaction product of (a) a reactive multi-arm polymer in which polymer arms of the reactive multi-arm polymer comprise a polymer segment having one end linked to a core region and an opposite end linked to a reactive moiety and (b) a hydrolytically stable iodinated multi-functional crosslinking compound which has plurality of complementary reactive moieties that is reactive with the reactive moieties of the reactive multi-arm polymer.

In some embodiments, the hydrogels break down in-vivo over a period ranging from 2 days or less to 80 weeks or more, for example, ranging anywhere from 2 days to 4 days to 1 week to 2 weeks to 4 weeks to 10 weeks to 20 weeks to 40 weeks to 80 weeks (i.e., between any two or the preceding time periods).

For example, a crosslinked reaction product can be formed from the following: a reactive multi-arm polymer having electrophilic groups and a multi-functional crosslinking compound having nucleophilic groups; a reactive multi-arm polymer having nucleophilic groups and a multi-functional crosslinking compound having electrophilic groups; a reactive multi-arm polymer having diene groups and a multi-functional crosslinking compound having dienophilic groups; a reactive multi-arm polymer having dienophilic groups and a multi-functional crosslinking compound having diene groups; a reactive multi-arm polymer having strained alkyne groups and a multi-functional crosslinking compound having azide groups; a reactive multi-arm polymer having azide groups and a multi-functional crosslinking compound having strained alkyne groups; a reactive multi-arm polymer having strained alkene groups and a multi-functional crosslinking compound having tetrazine groups; a reactive multi-arm polymer having tetrazine groups and a multi-functional crosslinking compound having strained alkene groups; a reactive multi-arm polymer having alkene groups and a multi-functional crosslinking compound having thiol groups; and a reactive multi-arm polymer having thiol groups and a multi-functional crosslinking compound having alkene groups.

In some embodiments, hydrolysable moieties that comprises a hydrolysable linker may be provided within the multi-functional crosslinking compound at positions internal to the complementary reactive moieties of the multi-functional crosslinking compound (e.g., wherein the complementary reactive moieties are each linked to a remainder of the multi-functional crosslinking compound through the hydrolysable linker).

In some embodiments, iodinated multi-functional crosslinking compounds may be employed to provide radiopacity. In some of these embodiments, the iodinated multi-functional crosslinking compounds are compounds that comprise two or more complementary reactive moieties that are reactive with the reactive moieties of the reactive multi-arm polymer and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

In some aspects of the present disclosure, systems are provided that are configured to deliver (a) multi-arm polymer as described herein and (b) a reactive iodinated multi-functional crosslinking compound as described herein. The multi-arm polymer and the iodinated reactive multi-functional crosslinking compound are co-mingled under conditions such that reactive moieties of the reactive multi-arm polymer react and form covalent bonds (e.g., glutaric ester linkages) with the complementary reactive moieties of the multi-functional crosslinking compound. Such systems can be used to form crosslinked hydrogels, either in vivo or ex vivo.

In some aspects of the present disclosure, systems are provided that comprise (a) a first composition that comprises a reactive multi-arm polymer as described herein and (b) a second composition that comprises a hydrolytically stable iodinated multi-functional crosslinking compound as described herein. For example, the first and second compositions can be first and second fluid compositions that, when the first and second fluid compositions are mixed, covalent bonds form between the hydrolytically stable reactive multi-arm polymer and the multi-functional crosslinking compound, resulting in a crosslinked reaction product of the reactive multi-arm polymer and the multi-functional crosslinking compound.

In some embodiments, systems are provided that comprise (a) a first composition containing the multi-arm polymer and the hydrolytically stable iodinated reactive multi-functional crosslinking compound and (b) a second composition comprising an accelerant that accelerates a crosslinking reaction between the hydrolytically stable reactive multi-arm polymer and the multi-functional crosslinking compound. For example, the first composition may be a fluid composition in which the reactive multi-arm polymer and the hydrolytically stable iodinated multi-functional crosslinking compound are intermixed under conditions where crosslinking is suppressed between the reactive moieties of the reactive multi-arm polymer and the complementary reactive moieties of the hydrolytically stable iodinated multi-functional crosslinking compound, and the second composition may be a fluid composition that, when mixed with the first fluid composition, causes covalent bonds to form between the reactive multi-arm polymer and the hydrolytically stable iodinated multi-functional crosslinking compound, resulting in a radiopaque crosslinked reaction product of the reactive multi-arm polymer and the multi-functional crosslinking compound. In certain embodiments, the accelerant in the second fluid composition changes the pH of the first fluid composition, resulting in crosslinking between the reactive multi-arm polymer and the hydrolytically stable multi-functional crosslinking compound.

The first composition may be a first fluid composition or may be first dry composition to which a suitable fluid such as water for injection, saline, etc. can be added to form a first fluid composition. The second composition may independently be a second fluid composition or may be second dry composition to which a suitable fluid such as water for injection, saline, etc. can be added to form a second fluid composition. The first and second compositions may independently be provided in vials, syringes, or other reservoirs.

The first and second compositions may further comprise additional agents, including therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a reactive multi-arm polymer as described above and a second reservoir that contains a second fluid composition that comprises a multi-functional crosslinking compound as described above. When the first and second fluid compositions are mixed, crosslinking occurs between the reactive multi-arm polymer and the hydrolytically stable multi-functional crosslinking compound.

In some embodiments, the system may include a delivery device that comprises a first reservoir that contains a first fluid composition that comprises the reactive multi-arm polymer and the hydrolytically stable multi-functional crosslinking compound, and a second reservoir that contains a second fluid composition that is an accelerant composition. The second fluid composition, when mixed with the first fluid composition, results in crosslinking between the reactive multi-arm polymer and the hydrolytically stable multi-functional crosslinking compound.

In either case, during operation, the first fluid composition and second fluid composition are dispensed from the first and second reservoirs and combined, whereupon the hydrolytically stable multi-functional crosslinking compound and the reactive multi-arm polymer and crosslink with one another to form a crosslinked hydrogel.

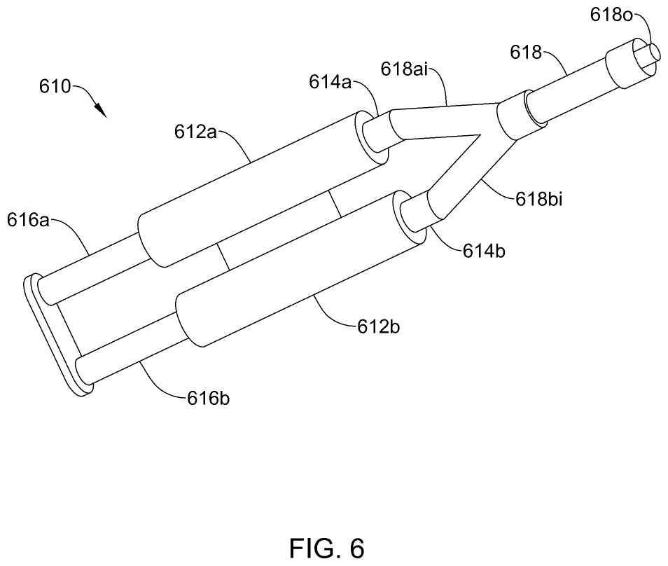

In particular embodiments, and with reference to FIG. 6, the system may include a delivery device 610 that comprises a double-barrel syringe, which includes a first barrel 612a having a first barrel outlet 614a, which first barrel contains the first fluid composition, a first plunger 616a that is movable in the first barrel 612a, a second barrel 612b having a second barrel outlet 614b, which second barrel 612b contains the second fluid composition, and a second plunger 616b that is movable in the second barrel 612b. In some embodiments, the device 610 may further comprise a mixing section 618 having a first mixing section inlet 118ai in fluid communication with the first barrel outlet 614a, a second mixing section inlet 618bi in fluid communication with the second barrel outlet, and a mixing section outlet 618o. Also shown are a syringe holder 622 configured to hold the first and second syringe barrels 612a, 612b, in a fixed relationship and a plunger cap 624 configured to hold the first and second plungers 616a, 616b in a fixed relationship.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an X-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

Crosslinked hydrogel compositions in accordance with the present disclosure include injectable fluid suspensions of crosslinked hydrogel particles.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

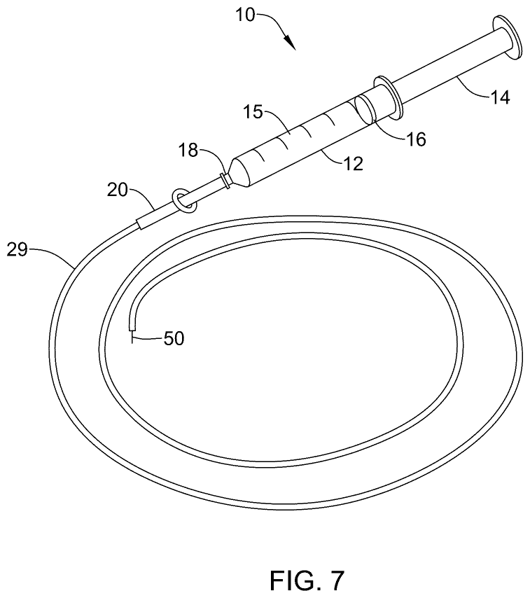

FIG. 7 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

It should be understood that this disclosure is, in many respects, only illustrative and that changes may be made in details without exceeding the scope of the disclosure. This may include, to the extent that it is appropriate, the use of any of the features of one embodiment being used in other embodiments.

What is claimed is:

1. A hydrolytically stable iodinated multi-functional compound comprising:
a multi-functional compound having functional groups that are reactive with a reactive group of a multi-arm polymer;
an iodinated polyol having reactive moieties including two or more hydroxyl groups, and one or more iodinated aromatic groups; and
a linker between a reactive moiety of the iodinated polyol and a functional group of the multi-functional compound, wherein the linker includes a carbamate linkage or a carbonate linkage.

2. The hydrolytically stable iodinated multi-functional compound of claim 1, wherein the linker has a hydrolytic stability in the presence of water that is greater than a hydrolytic stability of a comparative ester bond in the presence of water.

3. The hydrolytically stable iodinated multi-functional compound of claim 1, wherein the linker is a residue of a heteropolyfunctional linker, and wherein the linker is linked between a hydroxyl group of the iodinated polyol and a functional group of the functional groups of the multi-functional compound.

4. The hydrolytically stable iodinated multi-functional compound of claim 3, wherein the heteropolyfunctional linker is represented by the Formula 1:
$$( A ) m - R - ( B ) n ; \qquad ( Formula 1 )$$
where “R” is an organic segment;
where “A” is a first terminal functional group comprising a protected amine;
where “B” is a second terminal functional group comprising at least one of an isocyanate and a chloroformate ester;
where “m” is 1; and
where “n” is 1, 2, or 3.

5. The hydrolytically stable iodinated multi-functional compound of claim 3, wherein the heteropolyfunctional linker is a linear heterobifunctional linker.

6. The hydrolytically stable iodinated multi-functional compound of claim 5, wherein the linear heterobifunctional linker is a protected amino chloroformate.

7. The hydrolytically stable iodinated multi-functional compound of claim 6, wherein the protected amino chloroformate is selected from a group consisting of 4-(t-butoxycarbonylamino)butyl carbonochloridate, 2-(benzyloxycarbonylamino)ethyl carbonochloridate, 2-[2-(tbutoxycarbonylamino)ethyldisulfanyl]ethyl carbonochloridate, 3-(t-butoxycarbonylamino)propyl carbonochloridate, 2-[2-(tbutoxycarbonylamino) ethoxy]ethyl carbonochloridate, 6-(9H-fluoren-9-ylmethoxycarbonylamino)hexyl carbonochloridate, 2-(9H-fluoren-9-ylmethoxycarbonylamino)ethyl carbonochloridate, and chlorocarbonyl 2-(tritylamino)acetate.

8. The hydrolytically stable iodinated multi-functional compound of claim 3, wherein the heteropolyfunctional linker is a branched heteropolyfunctional linker.

9. The hydrolytically stable iodinated multi-functional compound of claim 8, wherein the branched heteropolyfunctional linker is a benzyloxycarbonyl (Cbz)-protected bis-chloroformate.

10. The hydrolytically stable iodinated multi-functional compound of claim 9, wherein the benzyloxycarbonyl (Cbz)-protected bis-chloroformate is: benzyloxycarbonyl(2-chlorocarbonyloxyethyl)amino]ethyl carbonochloridate or 6-[2-[benzyloxycarbonyl-[2-[6-chlorocarbonyloxyhexyl(methyl)carbamoyl]oxyethyl]amino]ethoxycarbonylmethyl-amino]hexyl carbonochloridate.

11. The hydrolytically stable iodinated multi-functional compound of claim 1, wherein the multi-functional compound is a polyamine.

12. The hydrolytically stable iodinated multi-functional compound of claim 1, wherein the multi-functional compound comprises trilysine acetate (TLA).

13. The hydrolytically stable iodinated multi-functional compound of claim 1, wherein the iodinated polyol is biocompatible iodinated contrast agent.

14. The hydrolytically stable iodinated multi-functional compound of claim 1, wherein the iodinated polyol is selected from a group including: iopromide, 5-Acetamido-N,N′-bis(2,3-dihydroxypropyl)-2,4,6-triiodoisophthalamide, Metrizamide, 1,3-Benzenedicarboxamide, 5-(acetylamino)-N1,N3-bis(2,3-dihydroxypropyl)-2,4,6-triiodo-N1-methyl- (ACI), iopamidol, Iomeprol, 1,3-Benzenedicarboxamide, N1,N3-bis[2-hydroxy-1-(hydroxymethyl)ethyl]-5-[(2-hydroxy-1-oxopropyl)amino]-2,4,6-triiodo- (ACI), ioxilan, isopentyl, ioversol, iohexol, iobitridol, iodixanol, and [1,3-Benzenedicarboxamide, 5,5′-[(1,3-dioxo-1,3-propanediyl)bis(methylimino)]bis[N,N′-bis[2,3-dihydroxy-1-(hydroxymethyl)propyl]-2,4,6-triiodo-(9CI, ACI)].

15. The hydrolytically stable iodinated multi-functional compound of claim 1, wherein the iodinated polyol is an azide-functionalized iodinated polyol including an azide functional group, and wherein the linker is formed between the azide functional group and a functional group of the functional groups of the multi-functional compound.

16. A radiopaque hydrogel formed by covalently crosslinking (a) a hydrolytically stable iodinated multi-functional compound comprising: a multi-functional compound having functional groups that are reactive with a reactive group of a multi-arm polymer; an iodinated polyol having reactive moieties including two or more hydroxyl groups, and one or more iodinated aromatic groups; and a linker between a reactive moiety of the iodinated polyol and a functional group of the multi-functional compound, wherein the linker includes a carbamate linkage or a carbonate linkage, with (b) a multi-arm polymer comprising a core, a plurality of polymer segments having a first end that is covalently attached to the core and a second end comprising a moiety that comprises a reactive group that is reactive with the functional groups of the hydrolytically stable iodinated multi-functional compound.

17. The radiopaque hydrogel of claim 16, wherein the reactive moieties of the multi-arm polymer include moieties that comprise electrophilic groups.

18. The radiopaque hydrogel of claim 17, wherein the electrophilic groups comprise succinimide ester groups; and wherein the plurality of polymer segments comprises polyethylene glycol segments.

19. The radiopaque hydrogel of claim 17, wherein the radiopaque hydrogel is formed in-vivo.

20. A kit for forming a radiopaque hydrogel comprising:
a reactive multi-arm polymer comprising a core, a plurality of polymer segments having a first end that is covalently attached to the core and a second end comprising a moiety that comprises a reactive group;
a multi-functional compound that comprises functional groups that are reactive with the reactive group of the reactive high molecular weight multi-arm polymer;
an iodinated polyol having two or more hydroxyl groups, and one or more iodinated aromatic groups; and
a heteropolyfunctional linker comprising a first terminal functional group that is reactive with a hydroxyl group of the iodinated polyol and at least one second terminal functional group that is reactive with the functional groups of the multi-functional compound to form a linker between the iodinated polyol and the multi-functional compound, wherein the heteropolyfunctional linker has is represented by Formula 1:
$$( A ) m - R - ( B ) n ; \qquad ( Formula 1 )$$
where “R” is an organic segment;
where “A” is a first terminal functional group comprising a protected amine;
where “B” is a second terminal functional group comprising at least one of an isocyanate and a chloroformate ester;
where “m” is 1; and
where “n” is 1, 2, or 3.
